(19) United States
(12) Patent Application Publication
(10) Pub. No.: US 2003/0003116 A1
(43) Pub. Date: Jan. 2, 2003

(54) Skin care agent and method of skin care

(75) Inventors: Yoshiharu Matahira, Shizuoka-ken (JP); Michiko Saito, Shizuoka-Ken (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: YAIZU SUISANKAGAKU INDUSTRY CO., LTD., Shizuoka-ken (JP)

(21) Appl. No.: 09/558,487

(22) Filed: Apr. 25, 2000

(30) Foreign Application Priority Data
Aug. 9, 1999 (JP) 225245/99

(51) Int. Cl. A61K 7/00

(52) U.S. Cl. 424/401

(57) ABSTRACT

The present invention provides a skin care agent comprising N-acetylglucosamine as an active ingredient, and a method of skin care using it. The promoter is preferably in the form of tablets, capsules, powder such as dust or granules, liquid or past. The skin care agent of the present invention may be incorporated into foods such as confectioneries, powdered soup and beverages. By orally ingesting the skin care agent of the present invention, the N-acetylglucosamine as an active ingredient is rapidly absorbed, and by utilizing a part thereof as a starting material of acidic mucopolysaccharides such as hyaluronic acid or chondroitin sulfate, the moisture and tension of skin can be improved and the rough skin and fine wrinkles can be prevented or ameliorated.

### FIELD OF THE INVENTION

The present invention relates to a skin care agent (or a skin-beautification promoter) which improves moisture and tension of skin and promotes prevention and amelioration of e.g. rough skin and fine wrinkles by orally ingesting it, and a method of skin care.

### BACKGROUND OF THE INVENTION

Acidic mucopolysaccharides such as hyaluronic acid or chondroitin sulfate have a high water retention, bond to collagen which serves as a column of intercellular substance matrix, and are mostly distributed in, for example, connective tissues, cartilaginous tissues and skin tissues, thereby being useful for keeping functions and forms of cells.

In the skin tissues, the acidic mucopolysaccharides, collagen, etc. mostly exist in corium layer and take a large part in water retention and resilience of skin. It is known that when the amounts thereof decrease due to aging or the like, the water retention and resilience of skin will be lost, thereby causing rough skin, fine wrinkles, etc.

Accordingly, in order to prevent and ameliorate the rough skin and fine wrinkles, it is important to maintain the moisture and tension of skin. For this purpose, cosmetics to which various components having effects for maintaining the moisture retention and resilience of skin are incorporated, are commercially available. As such components, for example, the mucopolysaccharides such as hyaluronic acid, chondroitin sulfate and collagen, low molecular weight saccharides such as trehalose and sorbitol, vitamins, amino acid derivatives, ceramide, α-orizanol, and fats and oils such as refined camellia oil, may be mentioned. Particularly recently, components derived from natural substances having a high safety are likely to be regarded as more worthy.

Further, many health and beauty care foods have been developed which enhance the above-mentioned effects by oral ingestion. For example, health and beauty care foods comprising nucleic acid and mucopolysaccharides which contain hyaluronic acid, chondroitin sulfate and collagen (Japanese Unexamined Patent Publication No. 10-165138), processed foods comprising as a main component a mixture of at least two food materials of active oxygen elimination factors, antiallergic factors, factors for improving e.g. skin, and antioxidation factors (Japanese Unexamined Patent Publication No. 10-70), foods comprising conchiolin or its processed product (Japanese Unexamined Patent Publication No. 8-173091), health foods comprising conjugated mucopolysaccharide wherein a mucopolysaccharide and peptide are bonded (Japanese Unexamined Patent Publication No. 9-98739), and health foods containing ceramide (Japanese Unexamined Patent Publication No. 11-113530), may be mentioned.

On the other hand, N-acetylglucosamine is one of natural aminosugars obtainable by decomposing a high molecular weight polysaccharide chitin contained in shells of crustacea such as crab and shrimp or lobster, and is a white crystalline powder having a good sweetness of about a half of sugar and being less in moisture absorption. N-acetylglucosamine is also contained in milk in a free state in an amount of about 10 mg/100 ml, and exists universally in living organisms as constituting units of sugar chains of mucopolysaccharide, glycoprotein and glucolipide. N-acetylglucosamine is usually produced from glucose as a starting material by metabolism in living organisms, and one of living organism components having a high safety to human being. As physiological actions of N-acetylglucosamine, amelioration of arthritis symptom, propagation-accelerating effect of Lactobacillus vifidus, and the like, have been known.

However, since hyaluronic acid, chondroitin sulfate, collagen and the like are high molecular weight compounds and hardly absorbed only by coating them on the skin like cosmetics, these components are used for the purpose of improving the water retention of skin surface when these are used for cosmetics. This is true for most of the above-mentioned other components. Further, if the high molecular weight compounds such as hyaluronic acid, chondroitin sulfate and collagen, are orally ingested, there is a problem in digestion and absorption and their effects are not necessarily satisfactory.

### SUMMARY OF THE INVENTION

Accordingly, it is an object of the present invention to provide a skin care agent which has an action such as improvement of moisture and tension of skin and prevention and amelioration of e.g. rough skin and fine wrinkles by oral ingestion, and a method of skin care.

The present inventors have intensively studied to accomplish the above object, and as a result, have found that N-acetylglucosamine orally ingested is rapidly absorbed from intestine and reaches cutaneous layer, and in the cutaneous layer, it promotes biosynthesis of mucopolysaccharides such as hyaluronic acid, and accomplished the present invention.

Namely, the present invention provides a skin care agent comprising an effective amount of N-acetylglucosamine in an ingestible carrier. The present invention also provides a method of skin care for a human comprising administering an effective amount of a skin care agent comprising N-acetylglucosamine in an ingestible carrier.

According to the present invention wherein N-acetylglucosamine is incorporated in the skin care agent, when it is ingested, most of the N-acetylglucosamine are rapidly absorbed and a part thereof is utilized as a starting material of mucopolysaccharides such as hyaluronic acid or chondroitin sulfate, whereby the moisture and tension of skin can be improved and the rough skin and fine wrinkles can be prevented and ameliorated.

### BRIEF DESCRIPTION OF THE DRAWINGS

FIG. 1 is a graph showing the tissue distribution of radioactivity after administration of radioactivity-labelled N-acetylglucosamine.

### DESCRIPTION OF THE PREFERRED EMBODIMENTS

Hereinafter, the present invention will be described in further detail with reference to preferred embodiments.

In the present invention, as N-acetylglucosamine (hereinafter referred to as “NAG”), NAG obtainable by hydrolysis of natural polysaccharide chitins derived from shells of crustacea such as crab and shrimp or lobster with an acid or an enzyme (natural-type NAG), or NAG obtainable by acetylation by chemical synthesis of a D-glucosamine chlorate which is obtainable by complete acidic hydrolysis of chitin (chemically synthesized NAG), may be used.

For example, the above-mentioned natural-type NAG can be obtained by methods disclosed in Japanese Patent No. 1822027, etc.

In the present invention, NAG is contained as an active ingredient in the skin care agent in an amount of preferably from 0.1 to 99% by weight, more preferably from 1 to 50% by weight, most preferably from 35 to 50% by weight.

According to more preferred embodiments of the present invention, the skin care agent should preferably contain other components which have already been recognized to have a skin-beautification (or skin care) effect, for example, collagen, chondroitin sulfate, hyaluronic acid, vitamin C, vitamin B group, trehalose and ceramide. Among them, it is preferred to contain at least one selected from collagen, chondroitin sulfate and vitamin C. In this instance, as the proportional ratios of respective components, it is preferred that collagen is from 1 to 20 parts by weight, chondroitin sulfate is from 0.5 to 10 parts by weight and vitamin C is from 0.1 to 10 parts by weight, based on 1 parts by weight of NAG. Further, it is preferred that NAG is contained in an amount of from 0.1 to 38% by weight in the skin care agent.

According to this embodiment, since NAG and the component having the skin-beautification effect can be ingested together, synergistic effect of skin-beautification (or skin care) promotion can be expected. Further, even if the above other components co-exist, NAG causes no reaction such as coloring or decomposition.

The uptake of the skin care agent of the present invention is preferably from 0.1 to 15 g, more preferably from 0.3 to 5 g, most preferably from 0.5 to 1.5 g in terms of NAG per day for adult. If the uptake of NAG is less than 0.1 g, the skin-beautification effect can not be expected, such being undesirable. Further, if the uptake of NAG exceeds 15 g, there is a possibility of symptom such as pasty stool or diarrhea, such being undesirable. In this connection, as shown in the test examples as described below, safety is confirmed even if NAG is orally administered in an amount of 5 g per kg of body weight of rat.

The skin care agent of the present invention is preferably formed in the shape of tablets, capsules, powder, granules, liquid or past.

For example, the tablets are obtained by uniformly mixing the components having skin-beautification effect together with excipients such as lactose and starch, and tabletting the mixture by a pressure-type tabletting machine.

The powder and granules are obtainable by using the above mixture as it is or subjecting it to granulation.

The capsules are obtainable by uniformly dispersing NAG and the components having skin-beautification effect in fats and oils such as safflower oil, and then adding e.g. beeswax thereto to appropriately adjust the viscosity of slurry, followed by filling it into a soft capsule made of gelatin and glycerol as main components of encapsulating materials by a soft capsule filling machine.

Further, NAG has a solubility of 32% by weight in water of 25° C. and is confirmed not to show coloration or decomposition even if heat treated with pH of 2 to 8 at 100° C. for 1 hour, and has a stability in usual food processing without problem at all, whereby the skin care agent of the present invention can be added as a food material to foods. In the present invention, as a carrier for the skin care agent, foods such as confectioneries, powdered soups, dairy products and beverages, may, for example, be mentioned. As such foods, specifically, gum, candies, tabletted confectioneries, chocolate, jelly, cookies, snacks, corn potage soup, consomme soup, milk, pudding, yogurt, ice cream, lactic acid beverages, alcohol beverages, vitamin beverages, mineral beverages, coffee beverages, near water, nutrition drinks, and the like, may be mentioned.

When the skin care agent of the present invention is ingested as food, NAG should preferably be contained in an amount of from 0.1 to 15 g, more preferably from 0.3 to 5 g, per meal.

### PREPARATION EXAMPLE 1

### Preparation of NAG

4 kg of chitin was added to 12 liters of conc. hydrochloric acid and partial hydrolysis was carried out while stirring at 40° C. for 3 hours. After the completion of the hydrolysis, this was diluted with water of the same volume, and neutralized to pH 5.0 with a 25% sodium hydroxide solution. 500 g of activated carbon was added to this neutralized solution, and stirring was carried out for 30 minutes for decoloring, and then filtration was carried out with a filter paper and insolubles and the activated carbon were removed to obtain 42 liters of a filtrate. This filtrate was subjected to deionization by an ion exchange membrane electrodialyser to obtain about 20 liters of the deionized liquid. This deionized liquid contained about 1.7 kg of chitinoligosaccharide. To this chitinoligosaccharide-containing solution, 50,000 units of chitinase (manufactured by Sigma Co.) was added, and then an enzyme was permitted to react thereto at 45° C. for 50 hours to decompose the chitinoligosaccharide and form NAG. After deactivation of the enzyme by heating, undecomposed chitinoligosaccharide was removed by treatment with 1 kg of activated carbon, and then treatment with an ion exchange resin was carried out, followed by condensation and freeze drying to obtain 1.35 kg of NAG having a purity of 99.5%.

### TEST EXAMPLE 1

### Acute Toxic Test of NAG

To each of five males and five females of Wister rats (SPF), NAG was given by a single oral administration in an amount of 5,000 mg per kg of body weight. After the administration, these rats were bred for 14 days and observed, and no death was recognized. It was also recognized that the 50% lethal dose (LD₅₀) to the rats was at least 5,000 mg per kg of body weight.

### TEST EXAMPLE 2

### NAG Kinetics Study

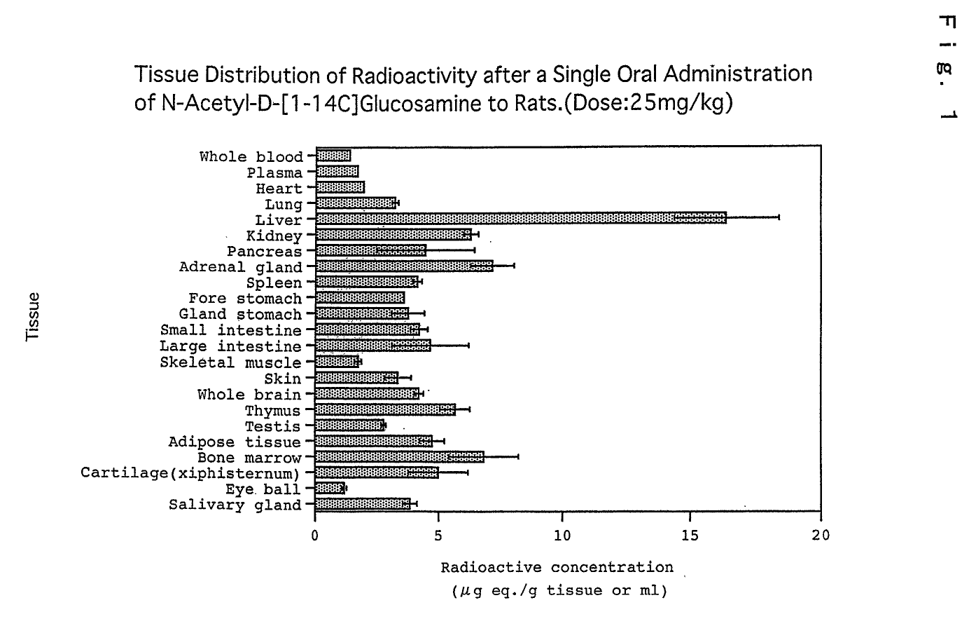

A mixture of ¹⁴C labelled NAG (1st-position carbon atom-labelled product: manufactured by Amasham Life Science Co.) and unlabelled NAG (the one prepared in PREPARATION EXAMPLE 1) was given to rats by single forced oral administration (250 mg/kg of body weight) for in vivo kinetics study. After the administration, the NAG was rapidly absorbed and the average concentration of radioactivity in blood reached the maximum value 4 hours after the administration and showed prompt attenuation until 24 hours later. About 60% of the administered NAG was utilized as an energy source and excreted as CO₂ to expiration. Further, about 20% was excreted to urine and stool. With respect to the residual of about 20%, the image analysis by autoradiography and the results of radioactive concentration analysis of the respective tissues as shown in FIG. 1, suggest that the residual of about 20% was widely transferred to e.g. cartilaginous tissues and fatty tissues in the living organism, and utilized as constituting substances of the living organism.

### TEST EXAMPLE 3

### Influential Test of NAG on Skin

Using hairless rats, influence of orally administered NAG on the content of hyaluronic acid in skin was tested. The NAG prepared in PREPARATION EXAMPLE 1 was mixed to a basic feed (Solid feed MF, manufactured by Oriental Kobo Kogyo Kabushiki Kaisha), and rats were permitted to freely ingest the mixture so that the substantial administered amount of NAG would be 0, 20 or 200 mg/kg of body weight/day. Administration was continued for 4 weeks from 9 week instar to 13 week instar, and the hyaluronic acid amounts in skin layer were measured separately for epidermis and corium. In this connection, the measurement of the hyaluronic acid was carried out in accordance with a hyaluronic acid measurement kit (manufactured by Chugai Shindanyaku Co.). The results are shown in Table 1.

TABLE 1

| | |
|---|---|
| | Hyaluronic acid content |
| Ingested amount | (μg/g dried tissue) |

| of NAG (mg/kg/day) | Epidermis | Corium |
|---|---|---|
| 0 | 31.25 | 462.7 |
| 20 | 33.77 | 506.6 |
| 200 | 35.09 | 549.8 |

As is evident from Table 1, it was confirmed that the hyaluronic acid contents in the epidermis and corium tend to increase together in proportion to the administered amounts of NAG.

### TEST EXAMPLE 4

### Influential Test of NAG on Skin

20 adult female volunteers of age ranging from 25 to 45 year old were classified into two groups i.e. a test group and a control group. They were asked to take tablets prepared similarly as in Example 1 as described below at a rate of 5 tablets per shots twice a day (NAG ingestion amount per day: 1.2 g) together with water, provided that the tablets for the control group were prepared by using lactose as a placebo instead of NAG. The test period was 60 days, and after the completion of test, they were questioned about skin conditions and the like by questionnaires. No particular restriction was made with respect to diet, makeup and the like during the test period. The results are shown in Table 2.

TABLE 2

| | Test | Placebo |
|---|---|---|
| Items of questionnaires | region | region |
| Feel moistness in skin | 7 | 2 |
| Feel tension in skin | 6 | 2 |
| Fine wrinkles decreased | 5 | 1 |
| Skin conditions improved as a whole | 8 | 2 |
| Skin conditions worsened as a whole | 0 | 1 |
| No change (including unidentified) | 2 | 5 |

As is evident from Table 2, as compared with the placebo region, many persons of the test region felt that the skin conditions were improved as a whole, for example, feeling moistness or tension in the skin as compared with the conditions before the start of the test. Accordingly, the skin-beautification effect of NAG was recognized.

### TEST EXAMPLE 5

To 22 females usually having a tendency of xeroderma and rough skin (average age: 25.5±10.7), a double blind long-term ingestion study was carried out in the following manners provided that a placebo of NAG-containing tablets were given to a control group.

Subject Group

NAG-containing tablets-administered group (NAG group, n=11): Tablets prepared similarly as in Example 1 as described below (NAG amount: 200 mg/tablet), were ingested at a rate of 5 tablets/day (NAG 1,000 mg/day, as a daily dose).

Placebo-administered group (Placebo group, n=11): Tablets prepared similarly provided that lactose was used instead of NAG, were ingested at a rate of 5 tablets/day.

Ingestion Period and Inspection Period

The ingestion-period was 8 weeks in each group. The inspection was in principle carried out just before the start of ingestion, 4 weeks after the start of ingestion, and just before the completion of ingestion (8 weeks after the start of ingestion).

Inspection Process

(1) Dermatologic Examination and Doctor's Questions

In the observation of whole body, 4 ranks evaluation (0: no symptom, 1: slight, 2: medium, 3: severe) was conducted with respect to pruritus, desiccation, flushing, erosion, desquamation, papula, vesicle and tumefaction. In the observation of face, 4 ranks evaluation was similarly conducted with respect to cosmetic dermatitis, desiccation, flushing and spread of cosmetics. Further, amelioration of general symptom including the observations of the whole body and face, was evaluated as a general observation. These evaluations were conducted by a plurality of doctors designated by Japan Dermatologic Science Society. The results are shown in Table 3 (average value of each score of each group).

TABLE 3

| | | Number of | | | |
|---|---|---|---|---|---|
| | | synpto- | Before | | |
| | | matic | inges- | After 4 | After 8 |
| | | persons | tion | weeks | weeks |
| | | NAG group (n = 11) | | | |
| Face | Cosmetic | 3 | 1.00 | 1.00 | 1.33 |
| | dermatitis | | | | |
| | Dessication | 11 | 2.00 | 1.18** | 1.00* |
| | Flushing | 10 | 1.80 | 1.10* | 1.10* |
| | Spread of | 6 | 1.83 | 1.33 | 0.83* |
| | cosmetics | | | | |
| Whole | Pruritus | 10 | 1.40 | 0.90 | 0.60 |
| body | Dessication | 11 | 2.09 | 1.36* | 1.00* |
| | Flushing | 5 | 1.40 | 0.80 | 0.40 |
| | Erosion | 1 | 2.00 | 1.00 | 0.00 |
| | Desquamation | 3 | 1.33 | 1.33 | 1.00 |
| | Papula | 2 | 1.00 | 1.50 | 1.50 |
| | Vesicle | 1 | 1.00 | 2.00 | 2.00 |
| General | | 11 | 1.82 | 1.27* | 1.09* |
| obser- | | | | | |
| vation | | | | | |
| | | Placebo group (n = 11) | | | |
| Face | Cosmetic | 0 | — | — | — |
| | dermatitis | | | | |
| | Dessication | 11 | 2.00 | 1.73 | 1.55 |
| | Flushing | 10 | 1.60 | 1.40 | 1.30 |
| | Spread of | 6 | 2.17 | 1.50 | 1.67 |
| | cosmetics | | | | |
| Whole | Pruritus | 5 | 1.80 | 1.60 | 1.00 |
| body | Dessication | 11 | 2.00 | 1.55* | 1.45* |
| | Flushing | 5 | 1.80 | 1.20 | 1.00 |
| | Erosion | 1 | 2.00 | 1.00 | 1.00 |
| | Desquamation | 2 | 1.00 | 1.00 | 1.00 |
| | Papula | 2 | 1.00 | 1.50 | 1.00 |
| | Vesicle | 1 | 1.00 | 1.00 | 2.00 |
| General | | 11 | 1.64 | 1.18 | 1.27 |
| obser- | | | | | |
| vation | | | | | |

Wilcoxon test: *:p<0.05, **:p<0.01

As shown in Table 3, with respect to the symptoms of face, in the NAG group, effects for significant improvements were recognized in the items of “dessication”, “flushing” and “spread of cosmetics”. On the other hand, in the placebo group, no significance was recognized in any item of observation. Further, in the symptom of whole body, significant improvements were recognized in the item of “dessication” in both groups. In the general observation, although significant improvements were recognized in the NAG group, no significant improvement was recognized in the placebo group.

(2) Moisture Content, Oil and Fat Content, and Acidity (pH)

The moisture content was measured by using Corneometer CM825 (manufactured by Courage+Khazaka Electronic Gmbh). This apparatus measures the moisture content of epidermis by measuring the electrostatic capacity via corneal layer, and is known to be less in error as compared with a conventional impedance method or infrared spectrophotometric method.

The oil and fat content was measured by using Sebumeter SM810 (manufactured by Courage+Khazaka Electronic Gmbh). In use of this apparatus, a special tape which absorbs only the oil and fat content is attached to the measurement site for 30 seconds and the oil and fat content is measured by the change of light transmittance of the tape. This apparatus is known not to be affected by humidity, etc.

The acidity was measured by using PH 900 (manufactured by Courage+Khazaka Electronic Gmbh). In use of this apparatus, an electrode is contacted to a skin surface through an ion-permeable membrane neighboring to a glass membrane and the acidity can be measured without electrochemical invasion. In this connection, the optimum pH of female's skin is about 5.5.

Measurement sites were 1 cm below the left eye, medial part of left upper arm (3 cm above the elbow), and poll (3 cm below spinous process of neck). With respect to the oil and fat content, since most subjects were scored at 0 at the left upper arm and the poll, only the site below the left eye was measured from the 2nd inspection.

In order to keep the environments for the measurements as equal as possible, a room having its internal conditions adjusted to be constant was prepared before the measurements (room temperature: 18 to 20° C., humidity: 45 to 60%), and the subjects were asked to keep in rest in this room for at least 30 minutes. Further, with respect to the makeup at the measurement site, it was in principle prohibited to put on makeup from 60 minutes before the inspection. The results are shown in Table 4.

TABLE 4

| | | Before | After 4 | After 8 |
|---|---|---|---|---|
| | | ingestion | weeks | weeks |
| | | NAG group (n = 11) | | |
| Moisture | Below left | 48.0 ± 8.8 | 58.8 ± 14.2* | 56.2 ± 8.4** |
| content | eye | | | |
| | Left upper | 37.8 ± 7.8 | 38.7 ± 5.8 | 36.2 ± 6.8 |
| | arm | | | |
| | Poll | 51.3 ± 5.6 | 51.9 ± 5.4 | 52.1 ± 10.9 |
| Acidity | Below left | 6.0 ± 1.0 | 5.7 ± 8.5 | 5.8 ± 0.4 |
| (pH) | eye | | | |
| | Left upper | 5.5 ± 0.3 | 5.6 ± 0.3 | 5.7 ± 0.4 |
| | arm | | | |
| | Poll | 5.7 ± 1.1 | 5.4 ± 0.3 | 5.3 ± 0.4 |
| Oil and | Below left | 63.8 ± 42.3 | 53.0 ± 29.2 | 40.8 ± 18.7* |
| fat | eye | | | |
| content | | | | |
| | | Placebo group (n = 11) | | |
| Moisture | Below left | 58.6 ± 11.7 | 57.8 ± 10.4 | 48.1 ± 10.2* |
| content | eye | | | |
| | Left upper | 37.6 ± 9.5 | 36.5 ± 6.7 | 32.2 ± 8.5 |
| | arm | | | |
| | Poll | 51.7 ± 18.9 | 49.2 ± 14.1 | 53.6 ± 20.8 |
| Acidity | Below left | 5.7 ± 0.6 | 5.7 ± 0.5 | 5.8 ± 0.5 |
| (pH) | eye | | | |
| | Left upper | 5.4 ± 0.4 | 5.7 + 0.4* | 5.6 ± 0.5 |
| | arm | | | |
| | Poll | 5.7 ± 0.7 | 5.5 ± 0.4 | 5.6 ± 0.4 |
| Oil and | Below left | 37.1 ± 32.2 | 30.1 ± 20.3 | 42.6 ± 30.1 |
| fat | eye | | | |
| content | | | | |

Wilcoxon test: *: p<0.05, **: p<0.01

As shown in Table 4, significant increase was confirmed in the moisture content at the site below the left eye in the NAG group. Further, significant decrease in the oil and fat content was confirmed. On the other hand, in the placebo group, significant decrease was recognized in the moisture content at the site below the left eye.

(3) Analysis by a Microscopic Three-Dimensional Skin Surface Analyzer (VISIONSCAN)

This analysis was conducted by using a digital analyzer of the skin surface (VISIONSCAN: manufactured by Courage+Khazaka Electronic Gmbh). In use of this apparatus, the skin surface was irradiated with special ultraviolet ray source, and the image is taken by a high performance CCD camera and digitalized for evaluation. The following factors were sampled as parameters.

(a) SEsm (Skin Smoothness)

This value is calculated from the average of the width and depth of wrinkles by the following formula (i), and one of indices which show the smoothness of skin. The smaller this value is, the smoother the skin surface is.

SEsm=(Co−Cu)*(Fmx−Fmy)*K (i)

Fmx: average width of furrows for the row analysis.

Fmy: average width of furrows for the column analysis.

Co: right frontier of the histogram whose calculation is based on a set-up values.

Cu: left frontier of the histogram whose calculation is based on a set-up values.

K: factor

(b) SEr (Skin Roughness)

This value is obtained by calculating the ratio of points darker than the set-up points in the whole image and further calculating by the following formula (ii), and one of indices which show the roughness of skin. The higher the value is, the rougher the skin is.

SEr=I/(Nx*Ny)*100 (ii)

I: counter whose start value is 0 and which is incremented each time the gray value of the current point is smaller than the threshold issued from the set-up programs.

Nx: amount of point per row.

Ny: amount of point per column.

(c) SEsc (Skin Scaliness)

Epidermolysis parts are counted to be brighter than the set-up values in the image. The SEsc value is obtained by calculating the ratio of these parts relative to the entire part by the following formula (iii), and is one of indices which show the dryness of scale (corneum). The lower the value is, the more moist and the less epidermolysis (scale).

SEsc=I/(Nx*Ny)*100 (iii)

I: counter whose start value is 0 and which is incremented each time the gray value of the current point is bigger than the threshold issued from the set-up programs.

Nx: amount of point per row.

Ny: amount of point per column.

(d) SEw (Skin Wrinkles)

This value is an index which is calculated by the following formula (iv) and shows the surface texture in vertical and horizontal directions of skin or the number and width of wrinkles. The higher the value is, the more the number of wrinkle is and the wider the width of wrinkles is.

SEw=Fmx*Fmy/(Fax*Fay)*Fay/Fax*K (iv)

Fax: amount of furrows for the row analysis.

Fmx: average width of furrows for the row analysis.

Fay: amount of furrows for the column analysis.

Fmy: average width of furrows for the column analysis.

K: factor

(e) Correction K (Kurtosis)

This value shows the smoothness of the whole skin. This value shows the quality of histogram in hue point of skin. The closer to 0 the value is, the smoother in curve the hue point is and the closer to ideal skin. The results of the above tests (a) to (e) are shown in Table 5.

TABLE 5

| | | Before | After 4 | After 8 |
|---|---|---|---|---|
| | | ingestion | weeks | weeks |
| | | NAG group (n = 11) | | |
| Kurtosis | Below left | 0.58 | 0.34 | 0.30 |
| (Ideal | eye | | | |
| value: 0) | Left upper | 0.97 | 0.40 | 0.50 |
| | art | | | |
| | Poll | 0.79 | 0.39 | 0.39 |
| SEsm | Below left | 378.1 | 337.6 | 309.4 |
| (Ideal | eye | | | |
| value: | Left upper | 384.7 | 324.7 | 379.9 |
| Lowest | art | | | |
| value) | Poll | 443.8 | 338.9* | 345.2* |
| SEr | Below left | 0.25 | 0.26 | 0.21 |
| eye | | | | |
| (Ideal | Left upper | 0.30 | 0.22 | 0.28 |
| value: | art | | | |
| Lowest | Poll | 0.51 | 0.32 | 0.40 |
| value) | | | | |
| SEsc | Below left | 238.4 | 137.0 | 133.0 |
| (Ideal | eye | | | |
| value: | Left upper | 342.9 | 195.8 | 202.4 |
| Lowest | art | | | |
| value) | Poll | 352.4 | 201.6 | 169.3* |
| SEw | Below left | | | |
| (Ideal | eye | 33.2 | 29.0 | 27.8 |
| value: | Left upper | 30.2 | 26.4 | 31.9 |
| Lowest | art | | | |
| value) | Poll | 27.3 | 23.4 | 29.5 |
| | | Placebo group (n = 11) | | |
| Kurtosis | Below left | 0.42 | 0.76* | 0.30 |
| (Ideal | eye | | | |
| value: 0) | Left upper | 0.43 | 0.59 | 0.42 |
| | art | | | |
| | Poll | 0.58 | 0.53 | 0.43 |
| SEsm | Below left | 379.6 | 398.8 | 353.7 |
| (Ideal | eye | | | |
| value: | Left upper | 335.7 | 373.1 | 335.1 |
| Lowest | art | | | |
| value) | Poll | 387.2 | 414.1 | 366.2 |
| SEr | Below left | 0.24 | 0.23 | 0.24 |
| (Ideal | eye | | | |
| value: | Left upper | 0.10 | 2.51 | 0.22 |
| Lowest | art | | | |
| value) | Poll | 0.35 | 0.33 | 0.44 |
| SEsc | Below left | 166.8 | 146.7 | 158.0 |
| (Ideal | eye | | | |
| value: | Left upper | 214.1 | 256.1 | 246.5 |
| Lowest | art | | | |
| value) | Poll | 231.8 | 219.7 | 202.2 |
| SEw | Below left | 29.8 | 33.6 | 33.7 |
| (Ideal | eye | | | |
| value: | Left upper | 23.7 | 28.1 | 24.4 |
| Lowest | art | | | |
| value) | Poll | 27.5 | 26.0 | 29.3 |

Wilcoxon test: *: p<0.05

As shown in Table 5, in the NAG group, significant decrease was confirmed in the SEsm value and the SEsc value at the poll, whereby it was found that the smoothness of skin was recovered, the dryness of corneum was reduced and the scale was decreased. On the other hand, in the placebo group, no significant decrease was confirmed in every value.

### EXAMPLE 1

Respective materials as indicated in Table 6 were mixed and granulated by a fluidized bed granulator, and then triangle tablets of 300 mg/tablet were formed by a tabletting machine (NAG content: 120 mg/tablet). The tabletting property was excellent.

TABLE 6

| | | |
|---|---|---|
| NAG | 40 | wt % |
| Collagen | 30 | wt % |
| Lactose | 15 | wt % |
| Cellulose powder | 10 | wt % |
| Citric acid | 2 | wt % |
| Perfume | 2 | wt % |
| Sucrose fatty ester | 1 | wt % |
| Total | 100 | wt % |

### EXAMPLE 2

Respective materials were kneaded so that the incorporated amounts per capsule (300 mg/capsule) would be as indicated in the following Table 7, and triangle soft capsules were prepared by a soft capsule filling machine by using gelatin as an encapsulating agent. The filling property was excellent.

TABLE 7

| | | |
|---|---|---|
| NAG | 30 | mg |
| Vitamin E | 50 | mg |
| Soybean lecithin | 20 | mg |
| Safflower oil | 170 | mg |
| Vitamin C | 20 | mg |
| Glycerol fatty ester | 5 | mg |
| Beeswax | 5 | mg |
| Total | 300 | mg/tablet |

### EXAMPLE 3

Respective materials were mixed in the proportion as indicated in Table 8 and granulation was carried out using a 0.5% guar gum solution as a binder by a fluidized bed granulator, to obtain 9.7 kg of NAG-containing granules. No moisture absorption of NAG was observed, dispersion of powder was good, and uniform granules were prepared.

TABLE 8

| | | |
|---|---|---|
| NAG | 1.5 | kg |
| Calcined cow bone powder | 0.8 | kg |
| Chondroitin sulfate | 0.3 | kg |
| Vitamin C | 0.1 | kg |
| Vitamin B mixture | 0.1 | kg |
| Glucose | 2.1 | kg |
| Dextrin | 3.5 | kg |
| Citric acid | 0.4 | kg |
| Lemon fruit juice powder | 1.2 | kg |
| Total | 10.0 | kg |

### EXAMPLE 4

All of respective materials were dissolved in water in the proportion as indicated in the following Table 9 to prepare a paste-like sample.

TABLE 9

| | | |
|---|---|---|
| NAG | 30 | g |
| Blue berry extract | 100 | g |
| Citric acid | 10 | g |
| White refined sugar | 50 | g |
| Pectin | 1 | g |
| Perfume | 0.2 | g |
| Water | 109 | g |
| Total | 300.2 | g |

This product was stable even after cold storage for one year.

### EXAMPLE 5

### Candy

Candy was prepared in accordance with a conventional method in the proportion as indicated in the following Table 10.

This candy could be prepared by usual steps without causing browning due to the addition of NAG.

TABLE 10

| | | |
|---|---|---|
| NAG | 20 | wt % |
| Sugar | 36 | wt % |
| Starch syrup | 40 | wt % |
| Fruit juice | 3 | wt % |
| Acidifier | 0.5 | wt % |
| Coloring matter, Perfume | 0.5 | wt % |
| Total | 100 | wt % |

### EXAMPLE 6

### Gummi

Gummi was prepared in accordance with a conventional method in the proportion as indicated in the following Table 11. The production of gummi includes a step of heating for evaporation of water after a step of mixing respective starting materials. Accordingly, in Table 11, the finished amount is less than the total of respective materials.

TABLE 11

| | | |
|---|---|---|
| NAG | 200 | g |
| Granulated sugar | 170 | g |
| Starch syrup | 260 | g |
| Sorbitol | 180 | g |
| Citric acid | 2 | g |
| Coloring matter, Perfume | 0.5 | g |
| Gelling agent | 80 | g |
| Water | 200 | g |
| Finished amount | 1,000 | g |

### EXAMPLE 7

### Cookie

Cookie was prepared in accordance with a conventional method in the proportion as indicated in the following Table 12.

This cookie could be prepared by usual steps without causing browning due to the addition of NAG.

TABLE 12

| | | |
|---|---|---|
| NAG | 80 | g |
| Unsalted butter | 120 | g |
| Sugar | 60 | g |
| Egg | 20 | g |
| Weak flour | 180 | g |
| Baking powder | 1 | g |
| Cocoa | 20 | g |
| Milk | 10 | g |

### EXAMPLE 8

### Jelly

Jelly was prepared in accordance with a conventional method in the proportion as indicated in the following Table 13. The production of jelly includes a step of heating for evaporation of water after a step of mixing respective starting materials. Accordingly, in Table 13, the finished amount is less than the water amount mixed with the starting materials.

TABLE 13

| NAG | 100 | g |
|---|---|---|
| Gelling agent | 3.5 | g |
| Sugar | 50 | g |
| Fruit juice | 10 | g |
| Perfume, Coloring matter | Proper quantity | |
| Acidifier, Sweetener | Proper quantity | |
| Water | 1,000 | ml |
| Finished amount | 750 | ml |

### EXAMPLE 9

### Powdered Soup

Powdered soup was prepared in accordance with a conventional method in the proportion as indicated in the following Table 14.

This powdered soup could be dissolved in hot water readily, and its taste was good.

TABLE 14

| | | |
|---|---|---|
| NAG | 4 | g |
| Chicken consomme | 0.5 | g |
| Dehydrated seaweed (Wakame) | 0.4 | g |
| Sesame oil | 0.1 | g |

150 ml of hot water/meal

### EXAMPLE 10

### Refreshing Drink

Refreshing drink was prepared in accordance with a conventional method in the proportion as indicated in the following Table 15.

TABLE 15

| | | |
|---|---|---|
| NAG | 1,000 | mg |
| collagen | 100 | mg |
| Ca lactate | 1,000 | mg |
| MgCl₂ | 50 | mg |
| Mixture of vitamins | 60 | mg |

| | |
|---|---|
| Acidifier, Perfume | Proper quantity |
| Sucrose, Glucose, Liquid sugar | Proper quantity |
| Preserver | Proper quantity |
| | 50 ml /bottle |

As described above, according to the present invention, it is possible to obtain a skin care agent by which rough skin and fine wrinkles can be prevented or ameliorated. By orally ingesting the skin care agent of the present invention, NAG is rapidly absorbed and transferred to skin layer, and then becomes a starting material of hyaluronic acid or the like, by which the moisture and tension of skin can be improved and the rough skin and fine wrinkles can be prevented or ameliorated.

What is claimed is:

1. A skin care-agent comprising an effective amount of N-acetylglucosamine in an ingestible carrier.

2. The skin care agent according to claim 1, which further comprises an effective amount of at least one selected from the group consisting of collagen, chondroitin sulfate, hyaluronic acid, vitamin C, vitamin B group, trehalose and ceramide.

3. The skin care agent according to claim 2, wherein the N-acetylglucosamine is contained in an amount of at least 0.1% by weight.

4. The skin care agent according to claim 1, wherein the N-acetylglucosamine is contained in an amount of at least 0.1% by weight.

5. The skin care agent according to claim 1, which further comprises at least one selected from the group consisting 1 to 20 parts by weight collagen, 0.5 to 10 parts by weight chondroitin sulfate, and 0.1 to 10 parts by weight vitamin C, based on 1 part by weight N-acetylglucosamine, and wherein the N-acetylglucosamine is contained in an amount of 0.1% to 38% by weight.

6. The skin care agent according to claim 1, wherein the skin care agent is in the form of tablets, capsules, powder, granules, liquid or paste.

7. The skin care agent according to claim 3, wherein the skin care agent is in the form of tablets, capsules, powder, granules, liquid or paste.

8. The skin care agent according to claim 1, wherein the ingestible carrier is food.

9. The skin care agent according to claim 8, wherein the ingestible carrier is a food selected from the group consisting of confectioneries, powdered soups, dairy products, beverages, gum, candies, tabletted confectioneries, chocolate, jelly, cookies, snacks, corn potage soup, consomme soup, milk, pudding, yogurt, ice cream, lactic acid beverages, alcohol beverages, vitamin beverages, mineral beverages, coffee beverages, near water and nutrition drinks, and wherein the N-acetylglucosamine is added to the food in an amount of 0.1% to 50% by weight

10. The skin care agent according to claim 8, wherein the N-acetylglucosamine is contained in an amount of from 0.1 to 15 g per portion of food.

11. A method of skin care for a human comprising administering an effective amount of a skin care agent comprising N-acetylglucosamine in an ingestible carrier.

12. The method of skin care according to claim 11, wherein the N-acetylglucosamine is administered in an amount of from 0.1 to 15 g per dosage.

13. The method of skin care according to claim 12, which further comprises administering an effective amount of at least one selected from the group consisting of collagen, chondroitin sulfate, hyaluronic acid, vitamin C, vitamin B group, trehalose and ceramide, contained in the ingestible carrier.

14. The method of skin care according to claim 2, wherein the N-acetylglucosamine is contained in the carrier in an amount of at least 0.1% by weight.

15. The method of skin care according to claim 13, wherein the ingestible carrier contains at least one selected from the group consisting 1 to 20 parts by weight collagen, 0.5 to 10 parts by weight chondroitin sulfate, and 0.1 to 10 parts by weight vitamin C, based on 1 part by weight N-acetylglucosamine, and wherein the N-acetylglucosamine is contained in an amount of 0.1% to 38% by weight.

16. The method of skin care according to claim 11, wherein the N-acetylglucosamine is contained in the carrier in an amount of at least 0.1% by weight and 0.1 to 15 g of the N-acetylglucosamine is administered.

17. The method of skin care according to claim 11, wherein the skin care agent is administered in the form of tablets, capsules, powder, granules, liquid or paste.

18. The method of skin care according to claim 12, wherein the skin care agent is administered in the form of tablets, capsules, powder, granules, liquid or paste.

19. The method of skin care according to claim 12, wherein the ingestible carrier is food and the dosage amount is a portion of the food.

20. The method of skin care according to claim 19, wherein the ingestible carrier is a food selected from the group consisting of confectioneries, powdered soups, dairy products, beverages, gum, candies, tabletted confectioneries, chocolate, jelly, cookies, snacks, corn potage soup, consomme soup, milk, pudding, yogurt, ice cream, lactic acid beverages, alcohol beverages, vitamin beverages, mineral beverages, coffee beverages, near water and nutrition drinks, and wherein the N-acetylglucosamine is added to the food in an amount of 0.1% to 50% by weight
